(19) United States
(12) Patent Application Publication
(10) Pub. No.: US 2025/0302368 A1
(43) Pub. Date: Oct. 2, 2025

(54) Wearable Device For Skin Testing

(71) Applicant: Shanghai Tianyin Biological Technology Co., Ltd., Shanghai (CN)

(72) Inventors: Xiaowen Hu, Shanghai (CN); Chunling Wan, Shanghai (CN)

(21) Appl. No.: 18/864,869

(22) PCT Filed: May 10, 2023
(86) PCT No.: PCT/CN2023/093176

§ 371 Date: Nov. 11, 2024

(30) Foreign Application Priority Data
May 18, 2022 (CN) 202210537553.5

(51) Int. Cl. A61B 5/00

(52) U.S. Cl. CPC A61B 5/4088; A61B 5/0077; A61B 5/4082; A61B 5/441; A61B 5/681; A61B 5/7271

(57) ABSTRACT

Provided is a wearable device for skin testing (100, 400), including: a main body (110, 410) including a first assembly part and a second assembly part; a liquid carrying apparatus (120, 420), the liquid carrying apparatus being assembled on the main body (110, 410) by means of the first assembly part, and having a reaction area (121) for contact with a wearer's skin to be tested; and an imaging apparatus (130, 430), the imaging apparatus being assembled on the main body (110, 410) by means of the second assembly part, and being configured to image the tested skin. The proposed wearable device (100, 400) for skin testing provides a small, convenient, and household skin testing scheme.

### TECHNICAL FIELD

The present application relates to the field of medical detection technology, and in particular to wearable devices for skin testing.

### BACKGROUND

As biological testing technology continues to evolve, a novel skin testing method has emerged to detect brain health abnormalities, such as certain mental and neurological diseases. This relies on the homology of cells in various tissues and organs of the human body; the cell structures of different tissues and organs share similarities. Therefore, the structural integrity of central nervous cells can be reflected by skin testing on the peripheral skin to some degree.

For example, the cell membrane system is not only the first line of defense to maintain cell integrity and resist external invasion, but also a signal transfer station for cells to communicate with the extracellular environment, and a material basis for signal transduction between cells of the nervous system. For healthy subjects, when a niacin reagent comes into contact with the skin surface, several biological processes involving the component of cellular plasma membrane take place, which will cause a transient redness and swelling reaction on the topical area of skin. However, when there are defects in the component of cellular membrane, the niacin-induced skin flushing reaction will be impaired and the redness and swelling reaction are abnormal; accordingly, the subject's nerve cells may have similar defects in the cellular membrane, which would contribute to diseases related to brain health. Therefore, a skin testing with niacin reagent (or a niacin derivative reagent) can be used to detect the brain health of the subject.

The test conditions required for the test involve multi-aspects, making the current tests too cumbersome. On the one hand, to ensure that the lighting and timing meet the requirements when imaging the tested skin, the testing equipment is very large in size and needs an enclosed chamber to accommodate the tested body parts. As such, individuals who intend to undergo testing have to visit a specialized testing institution to take the test, which makes the testing very inconvenient. Furthermore, some patients may give up visiting the testing institution for testing due to the sense of shame associated with the disease, thereby delaying diagnosis. On the other hand, the operation of existing testing equipment is extremely complicated, requiring trained professional operators for operation to ensure the test conditions meet the requirements, such as the contact time of the test reagent on the skin, the timing and lighting for imaging the tested skin, and so on.

### SUMMARY

In order to solve or at least alleviate one or more of the above problems or other existing problems, the present application provides the following technical solutions.

According to one aspect of the present application, there is provided a wearable device for skin testing. The wearable device comprises: a main body comprising a first assembly part and a second assembly part; a liquid carrying apparatus, the liquid carrying apparatus being assembled on the main body by means of the first assembly part, and having a reaction area for contact with a wearer's skin to be tested; and an imaging apparatus, the imaging apparatus being assembled on the main body by means of the second assembly part, and being configured to image the tested skin.

As an alternative or in addition to the above solution, the wearable device according to an embodiment of the present application further comprises a test strip component. The test strip component further comprises a mounting sheath and a test paper, wherein the mounting sheath is fitted onto the reaction area of the liquid carrying apparatus, and the test paper is connected to the mounting sheath, and is capable to carry a test reagent.

As an alternative or in addition to the above solution, in the wearable device according to an embodiment of the present application, the test strip component further comprises a limiting frame. The limiting frame is connected to the test paper and limits the contact area between the test paper and the skin to be tested.

As an alternative or in addition to the above solution, in the wearable device according to an embodiment of the present application, the liquid carrying apparatus is detachably assembled on the main body by means of the first assembly part; and the imaging apparatus is detachably assembled on the main body by means of the second assembly part. When the imaging apparatus is assembled on the main body by means of the second assembly part, a closed space is formed between the wearable device and the skin to be tested.

As an alternative or in addition to the above solution, in the wearable device according to an embodiment of the present application, the first assembly part and the second assembly part are the same part of the main body.

As an alternative or in addition to the above solution, in the wearable device according to an embodiment of the present application, the first assembly part and/or the second assembly part is a cavity.

As an alternative or in addition to the above solution, the wearable device according to an embodiment of the present application further comprises: a processing apparatus configured to determine a brain health level of a wearer based on imaging results from the imaging apparatus.

As an alternative or in addition to the above solution, in the wearable device according to an embodiment of the present application, the processing apparatus further comprises: an extraction unit configured to extract feature information associated with the brain health level of the wearer based on the imaging results; a comparison unit configured to compare the extracted feature information with a predetermined threshold; and a determination unit configured to determine the brain health level of the wearer based on comparison results from the comparison unit.

As an alternative or in addition to the above solution, in the wearable device according to an embodiment of the present application, the extraction unit is further configured to construct a color model based on the imaging results, and extract the feature information associated with the brain health level of the wearer based on image pixel color information in the color model.

As an alternative or in addition to the above solution, the wearable device according to an embodiment of the present application further comprises: an output apparatus configured to display the brain health level of the wearer determined by the processing apparatus; and/or a communication apparatus configured to send data outside the wearable device for processing or storing the data, wherein the data comprises the imaging results and/or the determined brain health level of the wearer.

As an alternative or in addition to the above solution, in the wearable device according to an embodiment of the present application, the brain health level of the wearer refers to the health level of the wearer concerning mental disorders and/or neurological diseases.

As an alternative or in addition to the above solution, in the wearable device according to an embodiment of the present application, the mental disorders at least include schizophrenia spectrum and other psychotic disorders, bipolar and related disorders, depressive disorders, anxiety disorders, trauma-and stressor-related disorders and neurodevelopmental disorders; and/or the neurological diseases at least include stroke, posterior circulation ischemia, Parkinson's disease, Parkinsonism, dementia and mild cognitive impairment.

As an alternative or in addition to the above solution, the wearable device according to an embodiment of the present application further comprises: a timing apparatus configured to start timing when the test reagent comes into contact with the skin to be tested.

As an alternative or in addition to the above solution, in the wearable device according to an embodiment of the present application, the timing apparatus further configured to issue a first instruction in response to the timing reaching a predetermined first time threshold, wherein the first instruction instructs issuing a reminder signal and/or instructs unloading the liquid carrying apparatus from the main body.

As an alternative or in addition to the above solution, in the wearable device according to an embodiment of the present application, the timing apparatus is further configured to issue a second instruction in response to the timing reaching one or more predetermined second time thresholds, wherein the second instruction instructs the imaging apparatus to image the tested skin.

As an alternative or in addition to the above solution, in the wearable device according to an embodiment of the present application, the test reagent is an HCAR2 receptor activator or an HCAR3 receptor activator or an ester thereof.

As an alternative or in addition to the above solution, in the wearable device according to an embodiment of the present application, the test reagent is niacin or a derivative thereof.

As an alternative or in addition to the above solution, in the wearable device according to an embodiment of the present application, the test reagent is a niacin reagent, a methyl nicotinate reagent, an ethyl nicotinate reagent, or a combination thereof.

The wearable device for skin testing according to the present application implements a small, convenient, and household skin testing scheme. Wearers can perform skin testing anytime and anywhere without having to visit a testing institution or seek a professional operator to perform testing.

### BRIEF DESCRIPTION OF THE DRAWINGS

The above and other purposes and advantages of the present application will become more complete and clearer with the following detailed description in conjunction with the drawings.

FIG. 1 shows an exploded view from a first perspective of a wearable device 100 for skin testing according to an embodiment of the present application.

FIG. 2 shows an exploded view from a second perspective of a wearable device 100 for skin testing according to an embodiment of the present application.

FIG. 3 shows an exploded view of a test strip component 140 of a wearable device 100 for skin testing according to an embodiment of the present application.

FIG. 4 shows a block diagram of a wearable device 400 for skin testing applicable to an embodiment of the present application.

### DETAILED DESCRIPTION OF EMBODIMENTS

It should be understood that, the terms “first”, “second” and so on in the specification and claims of the present application are used to distinguish similar objects during description, and are not necessarily used to describe a specific order or sequence. In addition, unless specifically stated otherwise, the terms “include”, “comprise”, “have” and similar expressions are intended to indicate non-exclusive inclusion.

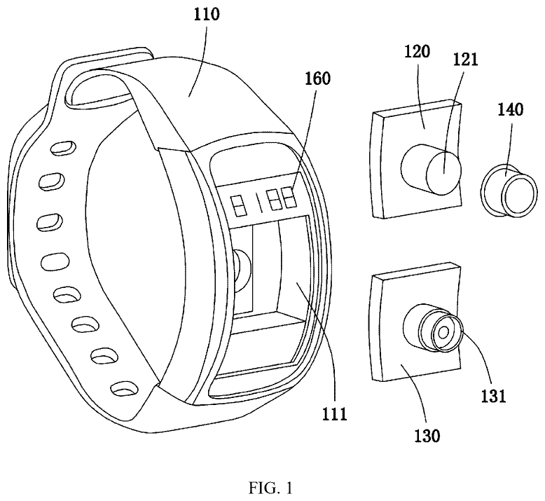

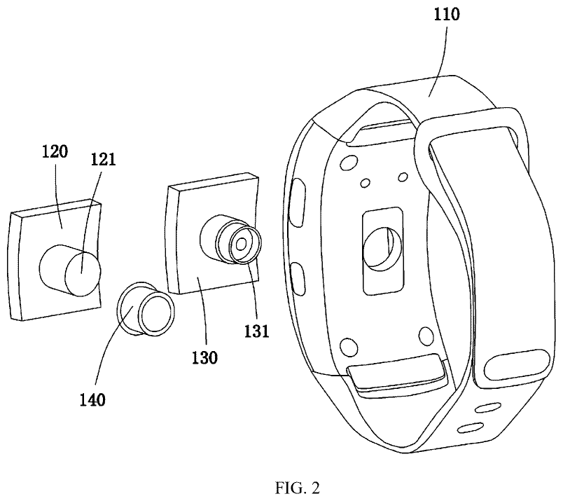

Hereinafter, various exemplary embodiments according to the present application will be described in detail with reference to the drawings. FIGS. 1 and 2 show front and back exploded views of a wearable device 100 for skin testing according to an embodiment of the present application. The wearable device 100 comprises a main body 110, a liquid carrying apparatus 120, and an imaging apparatus 130.

As illustrated in FIGS. 1 and 2, the main body 110 may comprise an assembly part 111 presented as a cavity. The liquid carrying apparatus 120 may be detachably assembled on the main body 110 by means of the assembly part 111, and the imaging apparatus 130 may also be detachably assembled on the main body 110 by means of the assembly part 111.

Among them, the liquid carrying apparatus 120 may have a reaction area 121 for contact with the wearer's skin to be tested, and the imaging apparatus 130 may be configured to image the tested skin. The imaging apparatus 130 may comprise an illumination portion 131, for example, provided in the form of a light ring. In the embodiment shown in FIGS. 1 and 2, when the imaging apparatus 130 is assembled on the main body 110 by means of the assembly part 111, a closed space can be formed between the wearable device 110 and the skin to be detected. This can provide a space with stable lighting for imaging. As an example, the imaging apparatus may be a CCD or COMS camera, a digital camera, etc., which captures the tested skin after skin testing into an image.

It should be noted that, in the context of the present application, the assembly part is not limited to the cavity form as illustrated in the embodiment of FIGS. 1 and 2, but may be any structural form capable of assembling the liquid carrying apparatus and the imaging apparatus on the main body.

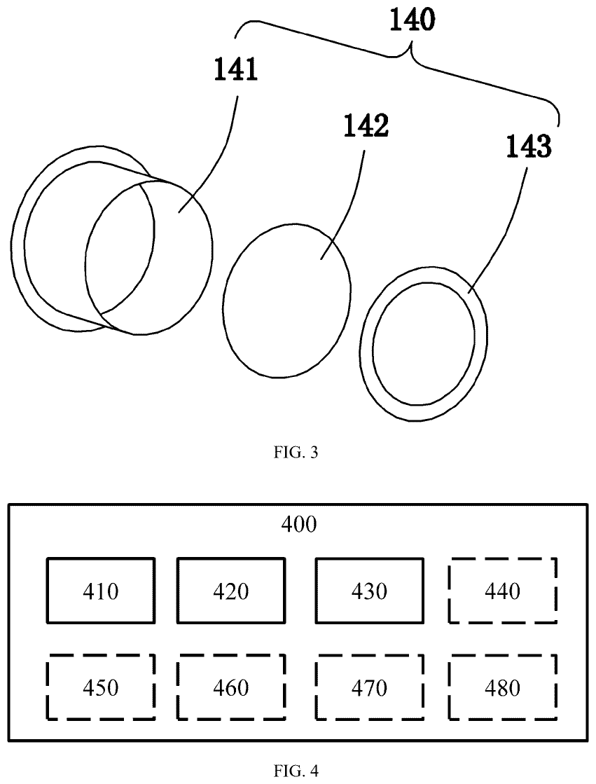

Further, the wearable device 100 may further comprise a test strip component 140. FIG. 3 shows an exploded view of the test strip component 140 of the wearable device 100. The test strip component 140 may further comprise a mounting sheath 141 and a test paper 142. The mounting sheath 140 is fitted onto the reaction area 121 of the liquid carrying apparatus 120. The test paper 142 is connected to the mounting sheath 141 and can carry a test reagent. When the test reagent is added to the test paper 142, the test strip component is mounted on the reaction area 121 of the liquid carrying apparatus 120, the liquid carrying apparatus 120 is assembled on the main body 110 and the wearable device 100 is worn, the test reagent comes into contact with the wearer's skin to be tested, thereby performing skin testing.

Moreover, the test strip component 140 further comprises a limiting frame 143. The limiting frame 143 is connected to the test paper 142 and limits the contact area between the test paper 142 and the skin to be tested. When the test reagent is added to the test paper 142, the limiting frame 143 can limit the contact area between the test reagent and the skin to be tested to a hollow reaction area of the limiting frame 143, so as to facilitate subsequent positioning operation when extracting the imaging results from the tested skin. In addition, when an excess amount of the test reagent is dropped onto the test paper 142, the limiting frame 143 can also guide the excess test reagent outside the hollow reaction area, to prevent the excess test reagent from affecting the result of skin testing.

Optionally, the test strip component 140 may be replaceable or disposable, to ensure accuracy and hygiene of each skin testing.

It should be noted that, in the embodiment shown in FIGS. 1 and 2, the liquid carrying apparatus 120 and the imaging apparatus 130 can be assembled on the main body 110 using the same assembly part 111, but the present application is not limited thereto. In other embodiments of the present application, the liquid carrying apparatus and the imaging apparatus may also be respectively assembled on the main body using different assembly parts, as long as they can respectively perform skin testing and imaging on the same area of the tested skin.

It should also be noted that, in the embodiment shown in FIGS. 1 and 2, both the liquid carrying apparatus 120 and the imaging apparatus 130 can be detachably assembled on the main body 110 of the wearable device 100, but the present application is not limited thereto. In other embodiments of the present application, the liquid carrying apparatus and the imaging apparatus may be fixedly assembled on the main body, rather than being detachable.

In the context of the present application, the “test reagent” is intended to mean a reagent that can be brought into contact with skin to perform skin testing. For example, the test reagent may be a reagent capable of determining the brain health level of a wearer. In the context of the present application, “the brain health level of a wearer” is intended to mean a health level concerning diseases that can be affected by the state of brain cells, for example, a health level concerning mental disorders and neurological diseases. Mental disorders may include schizophrenia spectrum and other psychotic disorders, bipolar and related disorders, depressive disorders, anxiety disorders, trauma-and stressor-related disorders and neurodevelopmental disorders (including intellectual developmental disorders, autism spectrum disorders, attention-deficit hyperactivity disorder, childhood communication disorders, specific learning disorders, tic disorders, movement disorders and other types of neurodevelopmental disorders) and other mental disorders affected by the state of brain cells. Neurological diseases may include stroke, posterior circulation ischemia, Parkinson's disease, Parkinsonism, dementia (including Alzheimer's disease, Lewy body dementia, Parkinson's disease dementia, frontotemporal lobar degeneration, vascular dementia and other types of dementia), mild cognitive impairment and other neurological diseases affected by the state of brain cells. As examples, the test reagent may be a HCAR2 receptor activator or a HCAR3receptor activator or an ester thereof, and the test reagent can be used for skin testing by activating the HCAR2 receptor or the HCAR3 receptor. Further, the HCAR2 receptor activator or HCAR3 receptor activator or an ester thereof may be niacin or a derivative reagent thereof, such as a niacin reagent, a methyl nicotinate reagent, an ethyl nicotinate reagent, or a combination thereof.

In the embodiment shown in FIGS. 1 and 2, the wearable device 100 is specifically in the form of a wristband. It can be readily appreciated by a person skilled in the art that in the context of the present application, the form of the wearable device is not limited thereto. It may also include any form of the wearable device fixed to the arm, leg, torso and other positions of the wearer, which enables the test reagent on the liquid carrying apparatus to come into contact with the skin to be tested to perform skin testing and enables the imaging apparatus to image the tested skin.

FIG. 4 shows a block diagram of a wearable device 400 for skin testing according to an embodiment of the present application. In this figure, the wearable device 400 comprises a main body 410, a liquid carrying apparatus 420, an imaging apparatus 430 and a processing apparatus 450. The main body 410 comprises a first assembly part and a second assembly part. The liquid carrying apparatus 420 is assembled on the main body 410 by means of the first assembly, and has a reaction area for contact with a wearer's skin to be tested. The imaging apparatus 430 is assembled on the main body 410, and is configured to image the tested skin.

Optionally, the wearable device 400 may further comprise a processing apparatus 450. The processing apparatus 450 can be used to determine the brain health level of the wearer based on imaging results from the imaging apparatus 430. Further, the processing apparatus 450 may comprise an extraction unit 451, a comparison unit 452 and a determination unit 453.

Among them, the extraction unit 451 is configured to extract feature information associated with the brain health level of the wearer based on the imaging results from the imaging apparatus 430. The feature information, for example, may be parameters such as color, area, perimeter or shape of a region of interest (e.g., a region reddened and swollen due to skin testing) in the imaging results.

Specifically, the extraction unit 451 is further configured to construct a color model based on the imaging results, and extract the feature information associated with the brain health level of the wearer based on image pixel color information in the color model. In other words, parameters such as color, area, perimeter or shape of the region of interest in the imaging results can be extracted based on, for example, the image pixel color information. In an example, when the constructed color model is an HIS color model, it is possible to determine the region of interest based on the H component, I component, S component or a combination thereof in the HSI color model as the image pixel color information, thereby extracting the parameters of the region of interest, that is, extracting feature information associated with the wearer's brain health level. In another example, when the constructed color model is a Lab color model, it is possible to determine the region of interest based on the L component, a component, b component or a combination thereof in the Lab color model as the image pixel color information, thereby extracting the parameters of the region of interest, that is, extracting feature information associated with the wearer's brain health level.

The comparison unit 452 is configured to compare the feature information extracted by the extraction unit 451 with a predetermined threshold. Here, the predetermined threshold for the feature information is a threshold for parameters such as color, area, perimeter or shape of a region of interest (e.g., a region reddened and swollen due to skin testing) in the imaging results. The threshold can be manually calibrated in advance or trained through machine learning or other methods.

The determination unit 453 is configured to determine the brain health level of the wearer based on comparison results from the comparison unit. For example, when the area of the reddened and swollen region extracted by the extraction unit 451 is smaller than a predetermined area threshold, the wearer's brain health level is determined as at risk of disease; when the area of the reddened and swollen region extracted by the extraction unit 451 is greater than a predetermined area threshold, the wearer's brain health level is determined as healthy.

In an embodiment, the method disclosed in patent CN202110707754.0 may be used for reference. In the extraction unit 451, the imaging results are scored for redness and swelling levels to serve as feature information; and in the comparison unit 452, the rated score is compared against a score threshold. Further, in the determination unit 453, the wearer's brain health level is determined based on the comparison results.

In specific applications, the processing apparatus 450 may be implemented by any feasible hardware such as chips, units, modules etc. Of course, it also allows for implementation through a combination of software and hardware.

The processing apparatus 450 may also be configured to perform analysis by synthesizing test results from multiple detections of the wearer, and compare the wearer's brain health level at different times or under other conditions.

The wearable device 400 may further comprise an output apparatus 460. The output apparatus 460 is used to output the wearer's brain health level determined by the processing apparatus 450. As an example, the output apparatus 460 may be a display 160 similar to that illustrated in FIG. 1, for presenting the wearer's brain health level in a visual form. As another example, the output apparatus may also be a speaker, for presenting the wearer's brain health level in the form of sound.

The wearable device 400 may further comprise a communication apparatus 470. The communication apparatus 470 is used to send data outside the wearable device 400 for processing or storing the data, for example, sending the data in a wired or wireless form to the outside, such as a local, remote or cloud-based storage, a processor, a server, etc. The data sent includes the imaging results from the imaging apparatus 430 and the brain health level of the wearer determined by the processing apparatus 450.

The wearable device 400 may further comprise a timing apparatus 380. The timing apparatus 380 is configured to start timing when the test reagent comes into contact with the skin to be tested. The time when the test reagent starts to contact the skin to be tested can be determined, for example, based on an output from the wearer or an operator of the wearable device 400, thus the timing starts. The time when the test reagent starts to contact the skin to be tested can also be determined, for example, based on the time when the liquid carrying apparatus 420 is assembled on the main body 410. That is, the time when the liquid carrying apparatus 420 is assembled on the main body 410 can be approximately determined as the time when the test reagent starts to contact the skin to be tested.

Further, the timing apparatus 380 may be configured to issue a first instruction in response to the timing reaching a predetermined first time threshold. The first instruction instructs issuing a reminder signal to remind the wearer or other operator of the wearable device 400 that the reaction time is over. Alternatively, the first instruction instructs unloading (e.g., ejecting) the liquid carrying apparatus 420 from the main body 410 (e.g., by means of a connection structure), so that the test reagent no longer contacts the skin, and the skin testing stops.

Among them, the first time threshold may be a predetermined time necessary for the test reagent to come in contact with the skin during skin testing, for example, 5 minutes, 10 minutes, 15 minutes, etc., and any other appropriate time predetermined according to practical skin testing requirements.

Further, the timing apparatus 380 may be configured to issue a second instruction in response to the timing reaching one or more predetermined second time thresholds. The second instruction instructs the imaging apparatus 430 to image the tested skin. The second time threshold may be a predetermined time necessary for capturing skin reaction images caused by skin testing, for example, 8 minutes, 12 minutes, 17 minutes etc., or any other appropriate time predetermined according to practical skin testing requirements. It can be readily appreciated by a person skilled in the art that the second time threshold is generally greater than the first time threshold, that is, the tested skin is imaged after the test reagent has completed contact with the skin. However, alternating contact and imaging is not excluded from the scope of the present application. For example, the timing starts when the skin to be tested comes into contact with a test reagent, the contact reaction occurs from 0 to 5 minutes, imaging is performed at the 7^{th} minute, another contact reaction occurs from the 8^{th} to 10^{th} minute, and imaging is performed again at the 12^{th} minute.

In alternative embodiments, when skin testing begins (i.e., the skin to be tested comes into contact with the test reagent), manual timing is started. When the reaction time reaches a first time threshold, an unloading instruction is manually issued to unload the liquid carrying apparatus from the wearable device 400, or the tested skin is manually separated from the test reagent. Similarly, the imaging apparatus may be instructed to perform imaging according to an input instruction (e.g., a manual instruction issued when the manual timing reaches a second time threshold).

In sum, the wearable device according to embodiments of the present application can provide a small, convenient, and household skin testing scheme. Wearers can perform skin testing by themselves anytime and anywhere to monitor their own brain health levels, without having to visit a testing institution or seek a professional operator to perform testing. This helps to timely detect relevant diseases, and makes it possible for timely intervention of the diseases. In addition, the wearable device according to embodiments of the present application can analyze and obtain the wearer's brain health level test results in real time, and can also compare the wearer's test results at different times and under different conditions.

It should be noted that some of the block diagrams illustrated in the drawings are only used to schematically represent functional entities, and do not necessarily correspond to physically or logically independent entities. These functional entities may be implemented in the form of software, or in one or more hardware modules or integrated circuits, or in different networks and/or processor devices and/or microcontroller devices.

It should also be understood that in some alternative embodiments, the functions/steps described may be performed in a different order than illustrated. For example, two functions/steps shown in sequence may be executed substantially at the same time or even in reverse order, depending on the specific functions/steps involved.

Although only some of the embodiments of the present application have been described above, it should be understood by a person skilled in the art that the present application can be implemented in many other forms without departing from the subject matter and scope thereof. Therefore, the examples and embodiments shown are deemed as illustrative rather than restrictive, and the present application may cover various modifications and substitutions without departing from the spirit and scope of the present application as defined by the appended claims.

1. A wearable device for skin testing, comprising:
a main body comprising a first assembly part and a second assembly part;
a liquid carrying apparatus, the liquid carrying apparatus being assembled on the main body by means of the first assembly part, and having a reaction area for contact with a wearer's skin to be tested; and
an imaging apparatus, the imaging apparatus being assembled on the main body by means of the second assembly part, and being configured to image the tested skin.

2. The wearable device according to claim 1, further comprising a test strip component, the test strip component further comprising a mounting sheath and a test paper,
wherein the mounting sheath is fitted onto the reaction area of the liquid carrying apparatus, and
the test paper is connected to the mounting sheath, and is capable to carry a test reagent.

3. The wearable device according to claim 2, wherein the test strip component further comprises a limiting frame, the limiting frame being connected to the test paper and limiting the contact area between the test paper and the skin to be tested.

4. The wearable device according to claim 1, wherein
the liquid carrying apparatus is detachably assembled on the main body by means of the first assembly part; and
the imaging apparatus is detachably assembled on the main body by means of the second assembly part,
wherein, when the imaging apparatus is assembled on the main body by means of the second assembly part, a closed space is formed between the wearable device and the skin to be tested.

5. The wearable device according to claim 1, wherein
the first assembly part and the second assembly part are the same part of the main body.

6. The wearable device according to claim 1, wherein
the first assembly part and/or the second assembly part is a cavity.

7. The wearable device according to claim 1, further comprising:
a processing apparatus configured to determine a brain health level of a wearer based on imaging results from the imaging apparatus.

8. The wearable device according to claim 7, wherein the processing apparatus further comprises:
an extraction unit configured to extract feature information associated with the brain health level of the wearer based on the imaging results;
a comparison unit configured to compare the extracted feature information with a predetermined threshold; and
a determination unit configured to determine the brain health level of the wearer based on comparison results from the comparison unit.

9. The wearable device according to claim 8, wherein the extraction unit is further configured to construct a color model based on the imaging results, and extract the feature information associated with the brain health level of the wearer based on image pixel color information in the color model.

10. The wearable device according to claim 7, further comprising:
an output apparatus configured to output the brain health level of the wearer determined by the processing apparatus; and/or
a communication apparatus configured to send data outside the wearable device for processing or storing the data,
wherein the data comprises the imaging results and/or the determined brain health level of the wearer.

11. The wearable device according to claim 7, wherein the brain health level of the wearer refers to the health level of the wearer concerning mental disorders and/or neurological diseases.

12. The wearable device according to claim 11, wherein the mental disorders at least include schizophrenia spectrum and other psychotic disorders, bipolar and related disorders, depressive disorders, anxiety disorders, trauma-and stressor-related disorders and neurodevelopmental disorders; and/or
the neurological diseases at least include stroke, posterior circulation ischemia, Parkinson's disease, Parkinsonism, dementia and mild cognitive impairment.

13. The wearable device according to claim 1, further comprising:
a timing apparatus configured to start timing when the test reagent comes into contact with the skin to be tested.

14. The wearable device according to claim 13, wherein
the timing apparatus further configured to issue a first instruction in response to the timing reaching a predetermined first time threshold,
wherein the first instruction instructs issuing a reminder signal and/or instructs unloading the liquid carrying apparatus from the main body.

15. The wearable device according to claim 14, wherein
the timing apparatus is further configured to issue a second instruction in response to the timing reaching one or more predetermined second time thresholds,
wherein the second instruction instructs the imaging apparatus to image the tested skin.

16. The wearable device according to claim 1, wherein the test reagent is an HCAR2 receptor activator or an HCAR3 receptor activator or an ester thereof.

17. The wearable device according to claim 16, wherein the test reagent is niacin or a derivative thereof.

18. The wearable device according to claim 17, wherein the test reagent is niacin, methyl nicotinate, ethyl nicotinate, or a combination thereof.
